(19) United States
(12) Patent Application Publication
(10) Pub. No.: US 2025/0011265 A1
(43) Pub. Date: Jan. 9, 2025

(54) COMPOSITIONS AND METHODS FOR PRODUCTION OF ANTIMICROBIAL AGENTS FROM BIO-RENEWABLE FEEDSTOCKS

(71) Applicant: SOLUGEN, INC., Houston, TX (US)

(72) Inventors: Shuai Qian, Houston, TX (US); Toni M. Lee, Missouri City, TX (US); Camille Boucher-Jacobs, Missouri City, TX (US); Brian Fisher, Houston, TX (US); Gaurab Chakrabarti, Houston, TX (US); Sean Hunt, Houston, TX (US); David Weiner, Houston, TX (US)

(73) Assignee: SOLUGEN, INC., Houston, TX (US)

(21) Appl. No.: 18/712,183

(22) PCT Filed: Feb. 22, 2023
(86) PCT No.: PCT/US2023/063051

§ 371 Date: May 21, 2024

Related U.S. Application Data
(60) Provisional application No. 63/312,402, filed on Feb. 22, 2022.

(51) Int. Cl. C07C 37/06; C02F 1/50; C02F 103/02; C12N 9/04

(52) U.S. Cl. CPC C07C 37/06; C02F 1/50; C12N 9/0006; C12Y 101/03009; C02F 2103/023; C02F 2303/04

(57) ABSTRACT

A chemoenzymatic method of producing a polyhydroxybenzene includes contacting glucose with one or more biocatalysts under conditions suitable for the formation of D-glucodialdose; and thermally treating D-glucodialdose in the presence of a dehydration catalyst under conditions suitable for the formation of the polyhydroxybenzene. A chemoenzymatic method of producing 1,2,3,4-tetrahydroxybenzene includes contacting glucose with a mutated copper radical oxidase and catalase under conditions suitable for the formation of D-glucodialdose; and subjecting D-glucodialdose and a dehydration catalyst to temperatures ranging from about 40° C. to about 100° C. suitable for the formation of 1,2,3,4-tetrahydroxybenzene.

### CROSS-REFERENCE TO RELATED APPLICATIONS

This application is a 35 U.S.C. § 371 national stage application of PCT/US2023/063051 filed Feb. 22, 2023 and entitled “Compositions and Methods for the Production of Antimicrobial Agents from Bio-Renewable Feedstocks,” which claims priority to U.S. Provisional Application No. 63/312,402 filed Feb. 22, 2022 and entitled “Compositions and Methods for the Production of Antimicrobial Agents from Bio-Renewable Feedstocks,” each of which is hereby incorporated herein by reference in its entirety for all purposes.

### STATEMENT REGARDING FEDERALLY SPONSORED RESEARCH OR DEVELOPMENT

Not applicable.

### REFERENCE TO SEQUENCE LISTING

The instant application contains a Sequence Listing which has been submitted electronically in XML file format and is hereby incorporated by reference in its entirety. Said XML file, created on Apr. 13, 2023, is named 3416-09902_21ENZ005_PCT Sequence Listing.xml and is 16,384 bytes in size.

### TECHNICAL FIELD

The present disclosure relates generally to compositions and methods for the production of antimicrobial and antioxidant agents. More particularly, the present disclosure relates to chemoenzymatic methods and compositions for the production of antimicrobial and/or antioxidant agents.

### BACKGROUND

The struggle of mankind against infectious diseases is well known. The discovery of antibiotics led to optimism that infections can be controlled and prevented. However, infections are still the leading cause of death in the developing world. This is due to the emergence of new diseases, the reemergence of diseases once controlled, and the appearance of antimicrobial resistance. It appears that the emergence of antimicrobial resistance is inevitable to almost every new drug, and it is recognized as a major problem in the treatment of microbial infections in both hospitals and the community at large.

Traditional methods for the production of antimicrobial and/or antioxidant agents may rely on biological pathways, such as fermentation, or standard petrochemical pathways using heat to generate a reaction. Both of these routes produce significant amounts of by-products which are disadvantageous to the economics of the process. Further, both of these routes result in the generation of large amounts of carbon dioxide (CO₂), either as a biological byproduct or as a product using heat to catalyze reactions. Thus, it would be desirable to develop methods and compositions having improved efficiencies and a reduced carbon footprint for the production of antimicrobial agents.

### BRIEF DESCRIPTION OF DRAWINGS

For a detailed description of the aspects of the disclosed processes and systems, reference will now be made to the accompanying drawings in which:

FIG. 1 is a schematic overview of aspects of the processes disclosed herein.

FIG. 2 is a graph of the specific activity of GAO mutants on glucose.

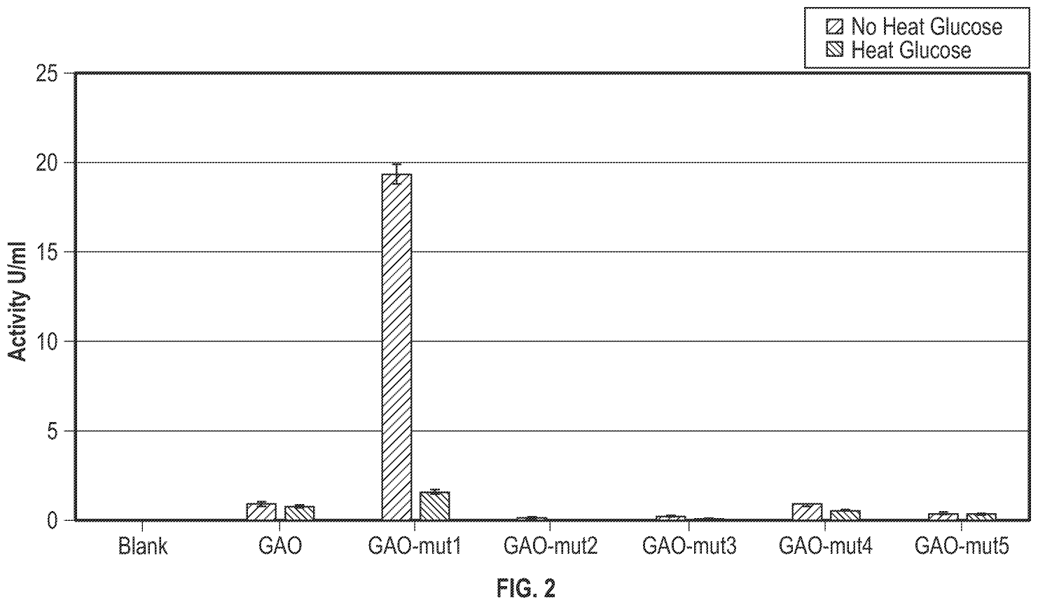

FIG. 3 is a graph of the specific activity of GAO-Mut47 and Mut107 on 0.5 and 2% glucose.

FIGS. 4 and 5 are graphs of the residual glucose concentration in parr reactions.

FIG. 6 depicts graphs of the integration peak area of GDA, reaction intermediates (mw. 176 g/mol and 174 g/mol), and the double dehydrated product (mw. 142 g/mol) on HPLC.

FIG. 7A depicts the TLC analysis of extracted dehydrated product (A).

FIG. 7B depicts the LC/MS analysis of purified fraction A and B from the TLC.

FIG. 8 is a plot of the 2D-NMR analysis (HSQC) of purified fraction A and B from TLC.

FIG. 9 depicts the FT-IR spectrum of purified fraction A and B from TLC analysis.

FIGS. 10 and 11 depict LC/QQQ MS analysis of fraction A and B with pyrogallol authentic standard.

FIG. 12 depicts a proposed reaction mechanism of pyrogallol and 1,2,3,4-tetrahydroxybenzene production from the GDA.

FIG. 13 depicts the HPLC integration peak area of GDA, reaction intermediates (mw. 176 g/mol and 174 g/mol), and 1,2,3,4-tetrahydroxybenzene from three different heat incubated GDA samples.

### BRIEF SUMMARY

A chemoenzymatic method of producing a polyhydroxybenzene comprising contacting glucose with one or more biocatalysts under conditions suitable for the formation of D-glucodialdose; and thermally treating D-glucodialdose in the presence of a dehydration catalyst under conditions suitable for the formation of the polyhydroxybenzene.

A chemoenzymatic method of producing 1,2,3,4-tetrahydroxybenzene comprising contacting glucose with a mutated copper radical oxidase and catalase under conditions suitable for the formation of D-glucodialdose; and subjecting D-glucodialdose and a dehydration catalyst to temperatures ranging from about 40° C. to about 100° C. to form 1,2,3,4-tetrahydroxybenzene.

### DETAILED DESCRIPTION

Disclosed herein are methods and compositions for the formation of antimicrobial and/or antioxidant agents from bio-based compounds. The methods disclosed herein utilize both a biocatalyst and a chemical catalyst and are referred to herein as chemoenzymatic methods. In one or more aspects of the present disclosure, the antimicrobial compound is a polyhydroxybenzene or derivatives thereof. Polyhydroxybenzene and derivatives thereof exhibit high potential as effective antimicrobial and/or antioxidant agents. In an aspect, the polyhydroxybenzene is 1,2,3,4-tetrahydroxybenzene (THB) that is also known as benzene-1,2,3,4-tetrol. THB has shown significant and broad-spectrum antimicrobial activity versus a wide range of gram positive, gram-negative bacteria, yeasts, and fungi, including pathogenic and non-pathogenic organisms. For simplicity, the compounds disclosed herein that may function as an antimicrobial and/or antioxidant is termed an active. Hereinafter the compositions disclosed herein are referred to as bio-based active agents (BAA).

In one or more aspects, the BAA comprises 1,2,3,4-tetrahydroxybenzene, alternatively the BAA consists essentially of 1,2,3,4-tetrahydroxybenzene. The conventional method for producing 1,2,3,4-tetrahydroxybenzene uses pyrogallol as the starting material where pyrogallol is converted to aminopyrogallol in a four-step synthesis. Aminopyrogallol is then hydrolyzed to give 1,2,3,4-tetrahydroxybenzene. Conversion of pyrogallol to 1,2,3,4-tetrahydroxybenzene requires the use of reagents such as phosgene, solvents such as pyridine and xylene, and forms a potentially explosive nitroaromatic as a synthetic intermediate.

Disclosed herein is a novel method of generating 1,2,3,4-tetrahydroxybenzene from glucose utilizing a biocatalyst, or alternatively a modified copper radical oxidase. Copper-radical oxidases (CROs) catalyze the two-electron oxidation of a large number of primary alcohols including carbohydrates, polyols and benzylic alcohols as well as aldehydes and α-hydroxy-carbonyl compounds while reducing molecular oxygen to hydrogen peroxide.

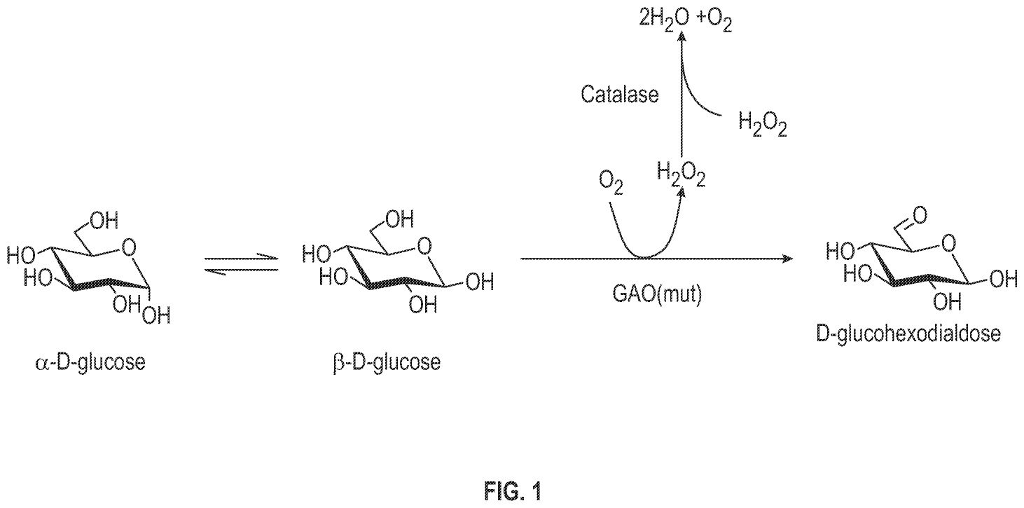

In one or more aspects and with reference to FIG. 1, a method of the present disclosure comprises the oxidation of glucose. In such aspects, glucose oxidation is catalyzed by an oxidase such as an oxidase engineered to accept glucose as a substrate. For example, and as depicted in FIG. 1, the oxidase is copper radical oxidase or alternatively a mutated galactose oxidase (mutGAO) which has been engineered to accept glucose as a substrate. In such aspects, the mutGAO oxidizes the C₆ alcohol to an aldehyde generating D-glucodialdose and hydrogen peroxide.

Herein, a modified copper radical oxidase suitable for use suitable for use in the present disclosure comprises a modified galactose oxidase (GAO, EC 1.1. 3.9). GAO is one of the most extensively studied alcohol oxidases with respect to both mechanistic investigations and practical applications. Other members in the copper radical oxidase family may be suitably employed in the present disclosure.

GAO is secreted by some fungal species, particularly Fusarium graminearum (also known as Gibbeella zeae), to aid in the degradation of extracellular carbohydrate food sources through catalyzing the oxidation of primary alcohols to aldehydes while generating hydrogen peroxide. The native function of GAO is the oxidation of D-galactose at the Ce position to generate D-galacto-hexodialdose. A small molecule (potassium ferricyanide) or auxiliary enzyme (i.e., horseradish peroxidase or HRP) is typically included to facilitate GAO activity. Typically, HRP is added to the reaction at a tenth of the weight percent (wt. %) of GAO. Although the GAO is promiscuous, the native form is unable to bind glucose due to steric clashes with F464 and F194 in the active site and the equatorial C₄ hydroxyl group on glucose. Efforts to engineer GAO to accept D-glucose as a substrate to form the C₆ aldehyde have resulted in improved activity as shown in Table 1. The M-RQW variant (R330K, Q406T, W290F) shows a specific activity of 1.6 U mg⁻¹. Another variant, the Des3-2 (Q326E, Y329K, R330K) showed four times higher activity on glucose than the native enzyme. In addition, the mutation C383S was found to improve catalytic efficiency up to three times by reducing the K_{M} of the enzyme on non-native substrates guar gum and methylgalactose through improved binding of the catalytic copper ion. Tables and 2 provide listings of some oxidase enzymes that may be useful in the methods of the present disclosure. Biocatalysts suitable for use in the present disclosure may have any of SEQ ID No. A through SEQ ID No.:9, for example, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, or combinations thereof.

TABLE 1

| Enzyme | | |
|---|---|---|
| Name | Mutations | Benefits |
| M-RQW | R330K Q406T W290F | 1.6 U/mg on glucose |
| Des3-2 | Q326E Y329K R330K | 4× activity on glucose vs WT |
| NA | C383S | Reduces K_{M} through improved |
| | | copper binding |
| M1 | S10P M70V P136 G195E | Improves E. coli expression |
| | V494A N535D | and solubility |
| N6M1 | A2A (GCC→GCA) S3S | Silent N-term mutations for |
| | (TCA→TCT) I5I | enhanced E. coli expression |
| | (ATC→ATT) | |
| NA | F194T C383E N245W/R | Improved specific activity of |
| | | up to 3 U/mg with N245R and |
| | | C383E in N6M1 background |
| NA | W290F R330K Q406T | 1.6 μM/min conversion on |
| | Y405F Q406E | glucose |

TABLE 2

| Name | Starting Mut | Additional mutations |
|---|---|---|
| M1 | wild-type | S10P M70V P136P |
| | | G195E V494A N535D |
| M-RQW | M1 | R330K Q406T |
| | | W290F I463P |
| GAO | M-RQW | C383S |
| GAO-mut1 | GAO | Y405F Q406E |
| GAO-mut2 | GAO | F194T |
| GAO-mut3 | GAO | C383E |
| GAO-mut4 | GAO | N245R |
| GAO-mut5 | GAO | Q326E Y329K |

In an aspect, biocatalysts suitable for use in production of a BAA of the type disclosed herein may further include one or more purified cofactors. Herein a cofactor refers to non-protein chemical compound that modulates the biological activity of the biocatalyst.

Many enzymes require cofactors to function properly. Nonlimiting examples of purified enzyme cofactors suitable for use in the present disclosure include thiamine pyrophosphate, NAD+, NADP+, pyridoxal phosphate, methyl cobalamin, cobalamine, biotin, Coenzyme A, tetrahydrofolic acid, menaquinone, ascorbic acid, flavin mononucleotide, flavin adenine dinucleotide, and Coenzyme F420. Such cofactors may be included in the biocatalyst preparation and/or be added at various points during the reaction. In some aspects, cofactors included with the biocatalyst preparation may be readily regenerated with oxygen and/or may remain stable throughout the lifetime of the enzyme(s).

As will be understood by one of ordinary skill in the art with the benefit of the present disclosure, reactions of the type disclosed herein may result in the production of byproducts (e.g., hydrogen peroxide) that can detrimentally impact other components of the reaction mixture. For example, hydrogen peroxide may degrade the biocatalyst resulting in a loss of catalytic activity. In such aspects, mitigation of the detrimental effects of hydrogen peroxide may be carried out such as by the introduction of a catalase (E.C. 1.11.1.61), the use of a hydrogen peroxide-resistant biocatalyst or combinations thereof.

In an aspect, a biocatalyst of the type disclosed herein is a wild type enzyme, a functional fragment thereof or a functional variant thereof. “Fragment” as used herein is meant to include any amino acid sequence shorter than the full-length biocatalyst, but where the fragment maintains a catalytic activity sufficient to meet some user or process goal. Fragments may include a single contiguous sequence identical to a portion of the biocatalyst sequence. Alternatively, the fragment may have or include several different shorter segments where each segment is identical in amino acid sequence to a different portion of the amino acid sequence of the biocatalyst but linked via amino acids differing in sequence from the biocatalyst. Herein, a “functional variant” of the biocatalyst refers to a polypeptide which has at one or more positions of an amino acid insertion, deletion, or substitution, either conservative or non-conservative, and wherein each of these types of changes may occur alone, or in combination with one or more of the others, one or more times in a given sequence but retains catalytic activity.

In the alternative or in combination with the aforementioned mutations, the biocatalyst may be mutated to improve the catalytic activity. Mutations may be carried out to enhance the protein or a homolog activity, increase the protein stability in the presence of substrates and products such as hydrogen peroxide and increase protein yield.

Herein, reference has been made to “sources” of biocatalysts. It is to be understood this refers to the biomolecule as expressed by the named organism. It is contemplated the biocatalyst may be obtained from the organism or a version of said biocatalyst (wildtype or recombinant) provided as a suitable construct to an appropriate expression system.

In an aspect, any biocatalyst of the type disclosed herein may be cloned into an appropriate expression vector and used to transform cells of an expression system such as E. coli, Saccharomyces sp., Pichia sp., Aspergillus sp., Trichoderma sp., or Myceliophthora sp. A “vector” is a replicon, such as plasmid, phage, viral construct or cosmid, to which another DNA segment may be attached. Vectors are used to transduce and express a DNA segment in cells. As used herein, the terms “vector” and “construct” may include replicons such as plasmids, phage, viral constructs, cosmids, Bacterial Artificial Chromosomes (BACs), Yeast Artificial Chromosomes (YACs) Human Artificial Chromosomes (HACs) and the like into which one or more gene expression cassettes may be or are ligated. Herein, a cell has been “transformed” by an exogenous or heterologous nucleic acid or vector when such nucleic acid has been introduced inside the cell, for example, as a complex with transfection reagents or packaged in viral particles. The transforming DNA may or may not be integrated (covalently linked) into the genome of the cell.

In an aspect, the gene of a biocatalyst disclosed herein is provided as a recombinant sequence in a vector where the sequence is operatively linked to one or more control or regulatory sequences. “Operatively linked” expression control sequences refers to a linkage in which the expression control sequence is contiguous with the gene of interest to control the gene of interest, as well as expression control sequences that act in trans or at a distance to control the gene of interest.

The term “expression control sequence” or “regulatory sequences” are used interchangeably and are used herein refer to polynucleotide sequences, which are necessary to affect the expression of coding sequences to which they are operatively linked. Expression control sequences are sequences that control the transcription, post-transcriptional events, and translation of nucleic acid sequences. Expression control sequences include appropriate transcription initiation, termination, promoter, and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “recombinant host cell” (“expression host cell”, “expression host system”, “expression system” or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

In one or more aspects, a method of the present disclosure further comprises the dehydration and rearrangement of D-glucodialdose to 1,2,3,4-tetrahydroxybenzene. The dehydration and rearrangement of D-glucodialdose to 1,2,3,4-tetrahydroxybenzene may be carried out by subjecting D-glucodialdose to temperatures ranging from about 40° C. to about 100° C., alternatively from about 80° C. to about 100° C., alternatively from about 60° C. to about 80° C. or alternatively from about 40° C. to about 60° C.

In one or more aspects, heat incubation of D-glucodialdose is carried out in the presence of a dehydration catalyst. Nonlimiting examples of catalysts that may facilitate the conversion of D-glucodialdose to 1,2,3,4-tetrahydroxybenzene include metals such as manganese, copper or polyanions such as phosphate. Advantageously, the amount of catalyst that efficiently facilitates the dehydration and rearrangement of D-glucodialdose may be so low as to be at levels attributable to impurities in the media utilized to carry out the reaction. For example, the dehydration and rearrangement of D-glucodialdose to 1,2,3,4-tetrahydroxybenzene may be carried out in the presence of catalyst amounts ranging from less than about 0.01 wt. % to about 1.0 wt. %, alternatively less than about 0.9 wt. %, alternatively less than about 0.8 wt. %, alternatively less than about 0.7 wt. %, alternatively less than about 0.6 wt. %, alternatively less than about 0.5 wt. %, alternatively less than about 0.4 wt. %, alternatively less than about 0.3 wt. %, alternatively less than about 0.2 wt. %, alternatively less than about 0.1 wt. %, alternatively less than about 0.05 wt. %, alternatively less than about 0.025 wt. %, or alternatively less than about 0.01 wt. %. In one or more alternative aspects, the dehydration and rearrangement of D-glucodialdose to 1,2,3,4-tetrahydroxybenzene may be carried out in the presence of catalyst that is added to the reaction mixture.

In one or more aspects, the method further comprises recovering 1,2,3,4-tetrahydroxybenzene. 1,2,3,4-tetrahydroxybenzene may be used without any further processing, Alternatively, 1,2,3,4-tetrahydroxybenzene may be purified using any suitable purification methodology such as simple distillation, centrifugation, crystallization, evaporation, chromatography, fractional distillation and combinations thereof.

In an aspect, the BAAs of this disclosure are included in a composition or formulation for administration to a subject. The terms “composition” and “formulation” as used herein refer to their generally accepted meanings in the art. These terms generally refer to forms in which an agent is suitable for administration (e.g., systemic or local administration) to a subject. Such compositions and formulations may comprise pharmaceutically acceptable diluents and/or pharmaceutically acceptable carriers.

The term “subject” as used herein, refers to an animal which is the object of treatment, observation, or experiment. By way of example only, a subject may be, but is not limited to, a mammal including, but not limited to, a human. In an aspect, the subject is administered the compositions disclosed herein in a therapeutically effective amount sufficient for treating, preventing, and/or ameliorating one or more symptoms of a disease or disorder. As used herein, amelioration of a symptoms of a disease or disorder by administration of a particular composition of the type disclosed herein refers to any lessening of that symptom, whether lasting or transient, which can be attributed to or associated with administration of compositions of the type disclosed herein.

In one or more aspects, the BAA is formulated for delivery to a material where bacterial growth is routinely mitigated. For example, the BAA may be introduced manually or automatically to a water source to mitigate the growth of microbial species. In an aspect, the BAA is introduced to industrial water. Herein, “industrial water” refers to water used in an industrial operation such as fabricating, processing, washing, diluting, cooling, or transporting a product; incorporating water into a product; or for sanitation needs. In an aspect, the industrial water is a feed water. Herein, a feed water refers to water used in boilers and cooling towers to ensure or enhance efficiency, maximize boiler and system life, reduce maintenance costs, maintain levels of operational performance, or the like. In one or more aspects, the industrial water is present in a cooling system such as a once-through cooling system, a closed recirculating cooling system, or dry cooling tower; or an open recirculating system such as a wet cooling tower or evaporative cooling tower. In another aspect, industrial water facilitates the cooling of a modern internal combustion engine.

In one or more aspects, a method of the present disclosure comprises the oxidation of glucose catalyzed by a mutated copper radical oxidase to form D-glucodialdose. D-glucodialdose in the presence of suitable catalyst and elevated temperatures may subsequently be dehydrated and rearranged to form 1,2,3,4-tetrahydroxybenzene. Advantageously, the methods disclosed herein may be carried out in a single reaction vessel which is commonly termed “one-pot synthesis.” One of ordinary skill in the art will readily recognize the benefits associated with one-pot synthetic processes such as simple operation, high mass efficiency, low cost, and a reduced amount of waste.

Disclosed herein are novel methods and compositions for the production of BAAs. In an aspect, the presently disclosed BAAs are produced at commercial scale and at purities of from about 70% to equal to or greater than about 90%, alternatively equal to or greater than about 70%, alternatively equal to or greater than about 80%, alternatively equal to or greater than about 90% with yields of from about 50% to about 90%, alternatively equal to or greater than about 50%, alternatively equal to or greater than about 60%, alternatively equal to or greater than about 70%, alternatively equal to or greater than about 80% or alternatively equal to or greater than about 90%.

### ADDITIONAL DISCLOSURE

A first aspect which is a chemoenzymatic method of producing a polyhydroxybenzene comprising contacting glucose with one or more biocatalysts under conditions suitable for the formation of D-glucodialdose; and thermally treating D-glucodialdose in the presence of a dehydration catalyst under conditions suitable for the formation of the polyhydroxybenzene.

A second aspect which is the method of the first aspect wherein the polyhydroxybenzene comprises 1,2,3,4-tetrahydroxybenzene.

A third aspect which is the method of any of the first through second aspects wherein the biocatalyst is a member of the copper radical oxidase family.

A fourth aspect which is the method of any of the first through third aspects wherein the one or more biocatalysts comprise a galactose oxidase.

A fifth aspect which is the method of any of the first through fourth aspects wherein the one or more biocatalysts comprise a mutated galactose oxidase.

A sixth aspect which is the method of any of the first through fifth aspects wherein the one or more biocatalysts has any of SEQ ID No.1 through SEQ ID No.:9.

A seventh aspect which is the method of any of the first through sixth aspects wherein the one or more biocatalysts comprises a catalase.

An eighth aspect which is the method of any of the first through seventh aspects wherein thermally treating comprises subjecting D-glucodialdose to a temperature ranging from about 40° C. to about 100° C.

A ninth aspect which is the method of any of the first through eighth aspects wherein the dehydration catalyst comprises copper, manganese, a polyanion or a combination thereof.

A tenth aspect which is the method of any of the first through ninth aspects further comprising recovering 1,2,3,4-tetrahydroxybenzene.

An eleventh aspect which is the method of any of the first through tenth aspects further comprising purifying 1,2,3,4-tetrahydroxybenzene.

A twelfth aspect which is the method of any of the first through eleventh aspects wherein the purity of 1,2,3,4-tetrahydroxybenzene ranges from about 70% to equal to or greater than about 90%.

A thirteenth aspect which is the method of any of the first through twelfth aspects wherein the yield of 1,2,3,4-tetrahydroxybenzene ranges from about 50% to about 90%.

A fourteenth aspect which is a chemoenzymatic method of producing 1,2,3,4-tetrahydroxybenzene comprising contacting glucose with a mutated copper radical oxidase and catalase under conditions suitable for the formation of D-glucodialdose; and subjecting D-glucodialdose and a dehydration catalyst to temperatures ranging from about 40° C. to about 100° C. suitable for the formation of 1,2,3,4-tetrahydroxybenzene.

A fifteenth aspect which is the method of the fourteenth aspect wherein the mutated copper radical oxidase comprises a galactose oxidase having any of SEQ ID No.7 through SEQ ID No.:9.

A sixteenth aspect which is the method of any of the fourteenth through fifteenth aspects wherein the purity of 1,2,3,4-tetrahydroxybenzene ranges from about 70% to equal to or greater than about 90%.

A seventeenth aspect which is the method of any of the fourteenth through sixteenth aspects wherein the yield of 1,2,3,4-tetrahydroxybenzene ranges from about 50% to about 90%.

An eighteenth aspect which is the method of any of the fourteenth through seventeenth aspects wherein the method is carried out in a single reaction vessel.

A nineteenth aspect which is the method of any of the fourteenth through eighteenth aspects further comprising contacting the 1,2,3,4-tetrahydroxybenzene with one or more a pharmaceutically acceptable carriers or pharmaceutically acceptable diluents.

A twentieth aspect which is the method of any of the fourteenth through nineteenth aspects further comprising introducing the 1,2,3,4-tetrahydroxybenzene to industrial water.

A twenty-first aspect which is the method of any of the fourteenth through twentieth aspects wherein the industrial water comprises feed water.

### EXAMPLES

The subject matter having been generally described, the following examples are given as particular aspects of the disclosure and are included to demonstrate the practice and advantages thereof, as well as aspects and features of the presently disclosed subject matter. It should be appreciated by those of skill in the art that the techniques disclosed in the examples which follow represent techniques discovered by the inventors to function well in the practice of the present subject matter, and thus can be considered to constitute suitable modes for its practice. However, those of skill in the art should, in light of the present disclosure, appreciate that many changes can be made in the specific aspects which are disclosed and still obtain a like or similar result without departing from the scope of the instant disclosure. It is understood that the examples are given by way of illustration and are not intended to limit the specification of the claims to follow in any manner.

### Example 1

Generation of GAO Mutant for Producing Glucodialdose from Glucose

A GAO was engineered to provide a mutant capable of converting glucose to glucodialdose. Following directed evolution and rational enzyme engineering, the improved GAO mutant exhibits a specific activity of >35 U mg⁻¹ on glucose.

### Directed Evolution

Directed evolution of thirty sites within 10 Å of the catalytic copper was performed on a parent sequence reported in Spadiut et al. containing the following added mutations: 1) R330, Q406T, W290F discovered by Sun et al. to introduce less than 1 U mg⁻¹ activity on glucose to GAO, 2) C383S discovered by Wilkinson et al. to lower the K_{M} of the enzyme on galactose, and 3) Y405F and Q406E discovered by Rannes et al. 2011 to enhance activity on a D-N-acetyl glucosamine substrate. Other mutations described in Table 3 were found to have neutral or deleterious effects on glucodialdose-generating activity. A new combination sequence was designated GAO-Mut1.

TABLE 3
Previously Described GAO Mutants with Reported
Activity on Glucose or Glucose Analog

| | | | |
|---|---|---|---|
| M1 | wild-type | Sun 2001⁷, | S10P M70V |
| | | Spadiut 2011¹¹ | P136P G195E |
| | | | V494A N535D |
| M-RQW | M1 | Sun 2002⁶ | R330K Q406T |
| | | | W290F I463P |
| GAO | M-RQW | Wilkinson 2004⁴ | C383S |
| GAO-mut1 | GAO | Rannes 2011¹⁰ | Y405F Q406E |
| GAO-mut2 | GAO | Chappell 2013⁹ | F194T |
| GAO-mut3 | GAO | Chappell 2013⁹ | C383E |
| GAO-mut4 | GAO | Chappell 2013⁹ | N245R |
| GAO-mut5 | GAO | Lippow 2010³ | Q326E Y329K |

Selected positions in GAO-Mut1 were mutated via the Quikchange method to all 20 amino acids using primers containing NNS codons. The constructs were then screened in the following manner Colonies were picked and used to inoculate one well each in a 96-well deepwell plate prefilled with lysogeny broth (LB). The grown clones were then used to inoculate autoinduction media in a separate 96-well deepwell plate for protein expression. Harvested cells were lysed with Bacterial Protein Extraction Reagent (B-PER) and the lysate was then screened for oxidase activity using a colorimetric (2,2′-azino-bis(3-ethylbenzothiazoline-6-sulfonic acid)) ABTS assay which detects hydrogen peroxide via a chromogenic enzyme-coupled reaction. Lysate was assayed for activity with and without exposure to heat. To assay activity in the absence of a heat challenge, lysate was diluted 50 times. A volume of 5 μL of the diluted lysate was combined with ABTS assay solution (final concentration of 2% w/v glucose, 0.0125 mg/ml horseradish peroxidase, 50 mM sodium phosphate buffer at pH 8, 0.05% ABTS) to a final volume of 200 μL and the change in absorbance at 405 nm was monitored until the reaction was complete. To assay residual activity after a heat challenge, 50 μL lysate was incubated for ten minutes at 50° C. and 20 μL of the heat-treated lysate was added to the ABTS solution before monitoring change in absorbance at 405 nm. Specific activity was calculated from the formulas below using the linear portion of the curve to measure ΔA405/min, taking the extinction coefficient of ABTS at 405 nm as 36.8 mM⁻¹·cm⁻¹, and dividing by two to account for the fact that one molecule of H₂O₂ oxidizes two molecules of ABTS in the HRP-catalyzed reaction.

$$Units {mg}^{- 1} = \frac{Δ {A}_{405} {min}^{- 1}}{36.8 \times 2 \times ( pathlength in cm ) \times ( mg enzyme ) / ( ml reaction mixture )}$$

$$Units {ml}^{- 1} = \frac{Δ {A}_{405} {min}^{- 1}}{36.8 \times 2 \times ( pathlength in cm ) \times ( ml enzyme ) / ( ml reaction mixture )}$$

Mutant lysates exhibiting a ΔA405/min greater than GAO-Mut1 were chosen for further characterization. Following identification of the mutation by DNA sequencing, hits were expressed, purified, and assayed for specific activity and thermostability as assessed by the temperature at which one half maximal activity was observed (T₅₀). Mutants were purified from 5 ml culture grown in auto-induction medium in a 24-well plate. Harvested cells were lysed with Bacterial Protein Extraction Reagent (B-PER) and the lysate was spun down with 15,000 rcf for 30 min at 4° C. The lysate supernatant was used for protein purification with HisPur™ Ni-NTA Spin Plates. The eluted protein sample was diluted with 100 mM potassium phosphate buffer pH 7.5 with 0.5 mM CuSO₄, and specific activity was measured using the ABTS assay outlined above. T₅₀ was measured by heating protein in the absence of substrate, cooling, and then measuring residual activity using the ABTS assay. Heating was accomplished by diluting the protein to a concentration of 2.5 mg/L in a volume of 100 mM phosphate buffer at pH 7.5, aliquoting 50 μL into a row of a 96-well PCR plate, and incubating over a temperature gradient for ten minutes. Promptly after heating, the mixture was cooled on ice and the ΔA405/min of 20 μL of enzyme solution in 200 μL of final volume of ABTS solution was measured as described above.

Hits were purified, tested for activity and T₅₀, and recombined to generate a final best mutant from the directed evolution step. Promising point mutants that could beneficially be combined in the Mut1 background included A193R, D404H, F441Y, and A172V (Table 4). These mutations were combined into a single combination mutant named GAO-Mut47 which exhibited a specific activity of 27.3 U mg⁻¹ and a T₅₀ of 56.8° C.

TABLE 4
T₅₀ and Specific Activity of Point and Combination
Mutants Identified via Directed Evolution

| | | | T₅₀ | Kcat | Km |
|---|---|---|---|---|---|
| Name | Mutations | U/mg | ° C. | S⁻¹ | mM |
| M-RQW-S | | 1.1 | 56.8 | 31.4 | 2168.3 |
| GAO-mut1 | Y405F Q406E | 14.0 | 51.8 | 30.2 | 93.1 |
| GAO-mut6 | Y405F Q406E S383C | 6.1 | 41.5 | 36.6 | 412.0 |
| GAO-mut7 | Y405F Q406E F441Y | 16.9 | 53.6 | 27.3 | 42.7 |
| GAO-mut8 | Y405F Q406E D404H | 15.0 | 53.7 | 30.7 | 83.9 |
| GAO-mut9 | Y405F Q406E G461A | 13.4 | 53.2 | 27.8 | 83.7 |
| GAO-mut10 | Y405F Q406E I462R | 12.1 | 53.2 | 31.6 | 130.7 |
| GAO-mut11 | Y405F Q406E A172V | 21.2 | 48.5 | 39.5 | 72.6 |
| GAO-mut12 | Y405F Q406E A193R | 15.4 | 56.3 | 28.0 | 64.8 |
| GAO-mut13 | Y405F Q406E A193T | 14.6 | 53.8 | 30.4 | 75.5 |
| GAO-mut14 | Y405F Q406E D404H | 18.8 | 55.0 | 26.5 | 29.7 |
| | F441Y | | | | |
| GAO-mut15 | Y405F Q406E G461A | 12.2 | 54.0 | 24.8 | 79.1 |
| | I462R | | | | |
| GAO-mut17 | Y405F Q406E D404H | 18.2 | 55.3 | 23.6 | 25.5 |
| | F441Y G461A I462R | | | | |
| GAO-mut18 | Y405F Q406E A193T | 18.1 | 56.6 | 24.1 | 28.0 |
| | D404H F441Y G461A | | | | |
| | I462R | | | | |
| GAO-mut19 | Y405F Q406E A193T | 13.3 | 46.3 | 24.5 | 70.8 |
| | D404H F441Y G461A | | | | |
| | I462R S383C | | | | |
| GAO-mut20 | Y405F Q406E A193T | 21.4 | 37.9 | 35.6 | 58.2 |
| | D404H F441Y G461A | | | | |
| | I462R S383C A172V | | | | |
| GAO-mut21 | Y405F Q406E F441Y | 18.3 | 53.8 | 24.2 | 29.6 |
| | G461A I462R | | | | |
| GAO-mut22 | Y405F Q406E A193T | 23.6 | 51.5 | 29.5 | 26.4 |
| | D404H F441Y G461A | | | | |
| | I462R A172V | | | | |
| GAO-mut23 | Y405F Q406E A193R | 21.1 | 57.5 | 27.2 | 26.8 |
| | D404H F441Y G461A | | | | |
| | I462R A172V | | | | |
| GAO-mut47 | Y405F Q406E A193R | 27.3 | 56.8 | 35.0 | 25.2 |
| | D404H F441Y A172V | | | | |
| GAO-mut58 | Y405F Q406E D404H | 27.1 | 52.9 | 35.4 | 26.6 |
| | F441Y A172V | | | | |

### Rational Engineering

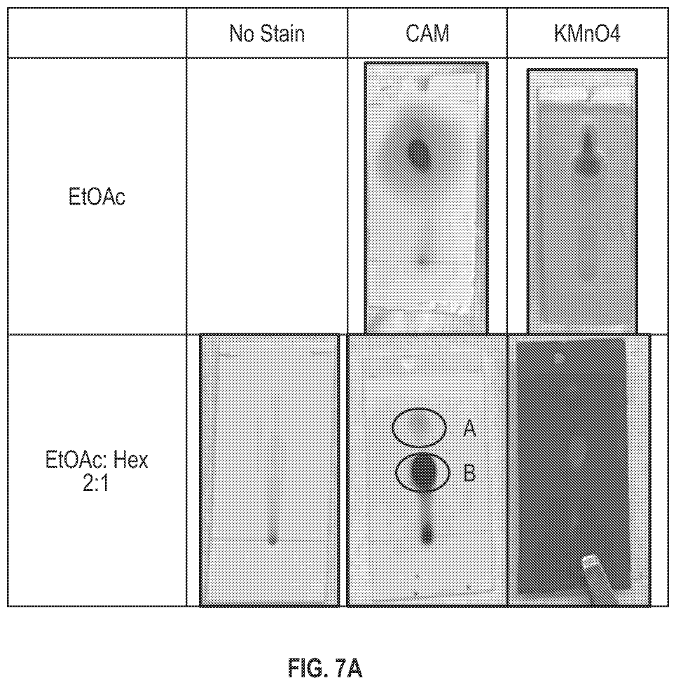

Rational engineering of GAO to further accept a glucose substrate and identify stabilizing mutations was accomplished with a combination of computational methods based on structural and multiple sequence alignment data (MSA). Previously, the inventors identified GAO-M-RQW S (the GAO-Mut1 sequence without the Y405F and Q406E mutations discovered by Rannes et al. 2011) as exhibiting the capability to accept both glucose and gluconate as substrate (FIG. 6). As efforts were underway to produce aGAO active on both substrates, rational design was performed on the GAO-M-RQW-S sequence rather than GAO-Mut1. Structural methods employed included applying FoldX12 (40 predicted mutations) and PROSS13 (80 mutations) to a modified form of the PDB structure 2WQ8 to contain the GAO-M-RQW-S mutations. MSA-based predictions were made using methods described by Sullivan et al. (34 mutations) and Komor et al. (28 mutations) applied to a 185-member MSA. This MSA was generated from an initial set of 1000 sequences curated with JALVIEW to remove sequences with 98% redundancy and retain only sequences experimentally verified as carbohydrate oxidases. 30 mutations identified by Merck in designing a GAO for synthesizing an intermediate of the HIV drug Islatravir were also added to the panel.

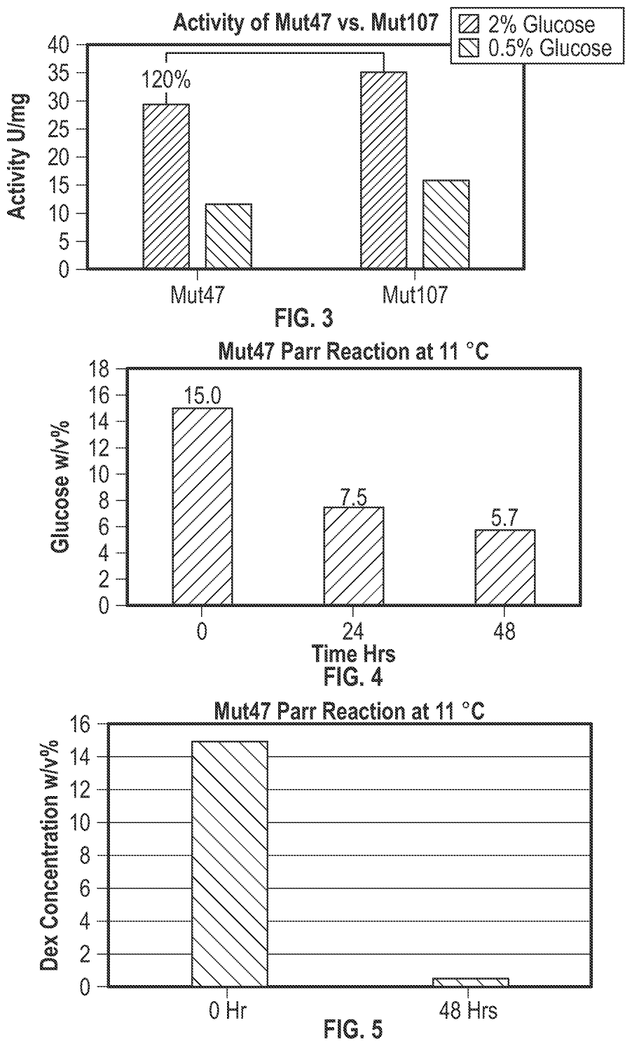

In total, 202-point mutants were screened using the same methods described above for screening the directed evolution clones. Thirty-nine hits were identified from an initial screen and sixteen were reidentified from a second round of screening. Upon generation of combo mutants in the best combination mutant from the directed evolution step (GAO-Mut47), the mutations N66S, S306A, S311F, and Q486L were identified as complementary and beneficial while N281, Y189W, S331R, A378D, and R459Q were deemed detrimental in this background (Table 5). The final GAO-Mut107 construct containing the Mut47 mutations and N66S, S306A, S311F, and Q486L exhibits a specific activity of 34.96 U mg⁻¹ on 2% glucose and a T₅₀ of 60.56° C. (FIG. 3). Additional mutations identified from machine learning algorithms were later incorporated to generate GAO-mut142 and GAO-mut164.

TABLE 5
Specific Activity and T50 of Rationally Designed Combination Mutants

| | | | | Fold | T50 |
|---|---|---|---|---|---|
| Clone | Mutations from Mut47 | New Mutations | U/mg | Improvement | ° C. |
| Mut47 | | | 31.11 | 1.00 | 57.64 |
| GAO-mut68 | N28I | N28I | 30.84 | 0.99 | 56.76 |
| GAO-mut69 | N28I N66S | N66S | 33.68 | 1.08 | 59.00 |
| GAO-mut70 | N28I N66S Y189W | Y189W | 31.80 | 1.02 | 59.91 |
| GAO-mut71 | N28I N66S Y189W S306A | S306A | 32.66 | 1.05 | 59.48 |
| GAO-mut72 | N28I N66S Y189W S306A | S311F | 33.87 | 1.09 | 60.81 |
| | S311F | | | | |
| GAO-mut73 | N28I N66S Y189W S306A | S331R | 27.56 | 0.89 | 59.87 |
| | S311F S331R | | | | |
| GAO-mut74 | N28I N66S Y189W S306A | A378D | 25.57 | 0.82 | 58.94 |
| | S311F S331R A378D | | | | |
| GAO-mut75 | N28I N66S Y189W S306A | R459Q | 23.51 | 0.76 | 58.49 |
| | S311F S331R A378D R459Q | | | | |
| GAO-mut76 | N28I N66S Y189W | V477D | 19.22 | 0.62 | 59.17 |
| | S306A S311F S331R | | | | |
| | A378D R459Q V477D | | | | |
| GAO-mut77 | N28I N66S Y189W S306A | Q486L | 24.57 | 0.79 | 59.88 |
| | S311F S331R A378D R459Q | | | | |
| | V477D Q486L | | | | |
| GAO-mut107^{a} | N66S S306A S311F Q486L | Removed N28I, | 34.96 | 1.20 | 60.56 |
| | | Y189W, S331R, | | | |
| | | A378D, R459Q, | | | |
| | | and V477D | | | |
| GAO-mut142^{b} | N66S S306A S311F Q486L | H40C | 37.53 | 1.29 | 58.76 |
| | H40C | | | | |
| GAO-mut164^{b} | N66S S306A S311F Q486L | L71C | 38.22 | 1.32 | 52.97 |
| | H40C L71C | | | | |

Bolded mutations are beneficial in a Mut47 background A193R D404H F441Y A172V
^{a}Data collected in a separate experiment from other data. Fold improvement is calculated compared to an internal Mut47 control
^{b}Data collected in a separate experiment from other data. Fold improvement is calculated compared to an internal Mut47 control.

One-Step Parr Bomb Reaction with GAO-Mut47 to Produce D-Glucodialdose

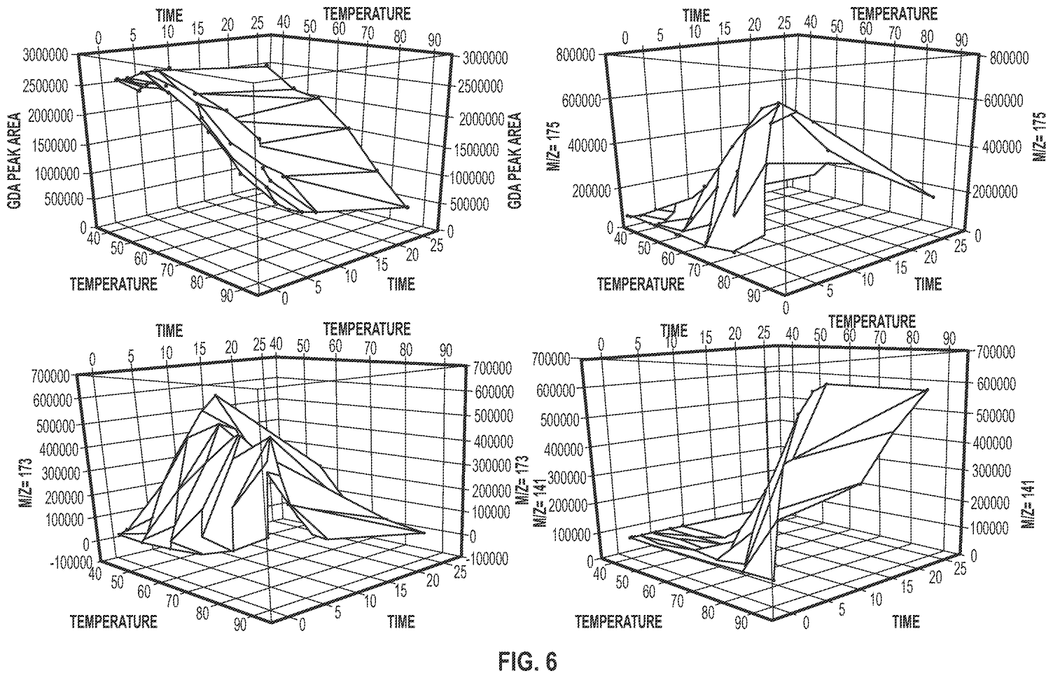

A 50 mL reaction was conducted in a 200 mL vessel pressurized to 100 psi. The vessel was charged with 50 mM sodium phosphate pH 8 buffer, 50 μM CuSO₄, 15 w/v % glucose, 0.005 w/v % catalase, 0.001% horseradish peroxidase, and 0.001 w/v % engineered GAO. The reaction was stirred at 500 rpm at 11° C. for 48 hours. Samples were taken at 0, 24, and 48 hours then assayed with high-performance liquid chromatography (HPLC) to measure residual glucose and the results are presented in FIG. 4.

### Example 2

Generation of Dehydrated GDA Product from Heat Incubation.
One-Step Parr Bomb Reaction with GAO-Mut47 to Produce D-Glucodialdose with High Yield.

A 100 mL reaction was conducted in a 400 mL vessel pressurized to 100 psi. The vessel was charged with 50 mM sodium phosphate pH 8 buffer, 50 μM MnSO₄, 15 w/v % glucose, 0.005 w/v % catalase, 0.001% horseradish peroxidase, and 0.01% engineered GAO. The reaction was stirred 500 rpm at 11° C. for 48 hours. Samples were taken at 0 and 48 hours then assayed with HPLC to measure residual glucose. After 48 hrs reaction, >95% of glucose was converted to GDA, the results are shown in FIG. 5.

### Heat Incubation of GDA Solution to Catalyze the Dehydration Reaction

100 μL of GDA solution that was generated from the parr reaction (˜14.5 w/v % of GDA, 0.5% w/v % of glucose) was aliquoted into each well of the PCR plate, and the samples were heat incubated at a range of temperatures from 40 to 90° C. Sample were taken at time 0, 0.5, 1, 2, 4, 6, 8, and 24 hrs then assayed with HPLC to monitor the formation of dehydrated product, 1,2,3,4-tetrahydroxybenzene, and other intermediate products.

The results were analyzed by HPLC. As shown in FIG. 6, GDA degradation is observed when the incubation temperature is higher than 50° C., and the degradation percentage increases by increasing the incubation temperature or elongating the incubation time. After 24 hrs incubation at 90° C., the GDA degradation percentage is about 90%. With short incubation time around 10 hrs, two intermediate compounds with molecular weight of 176 and 174 were generated in the incubation reaction. After increasing the incubation time to 24 hrs, the reaction yielded the 1,2,3,4-tetrahydroxybenzene (142 g/mol) as the major product.

### Example 3

Characterization of the Generated 1,2,3,4-tetrahydroxybenzene.

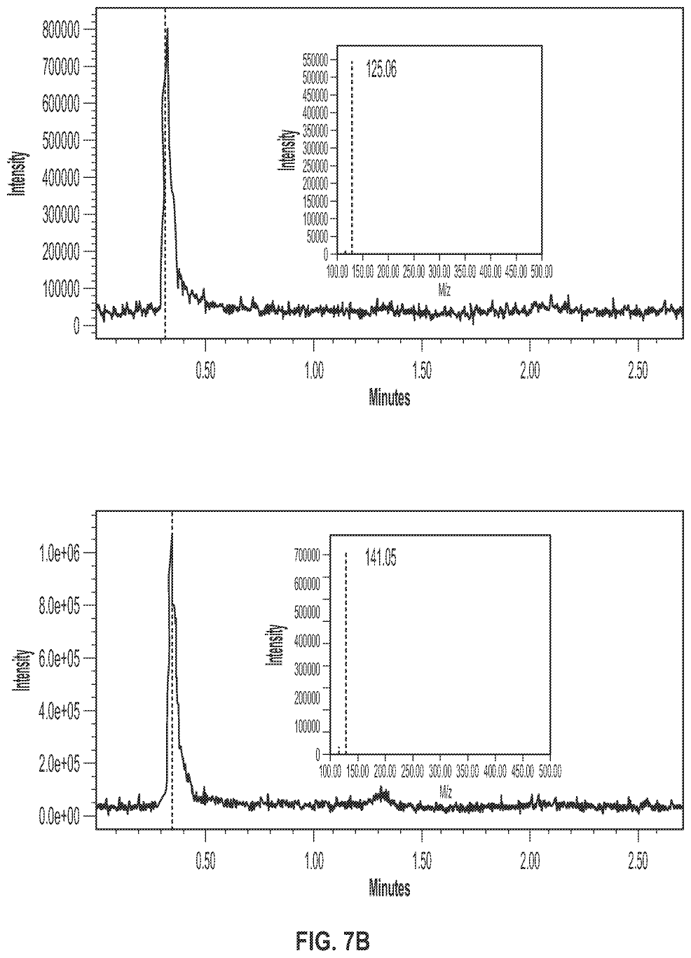

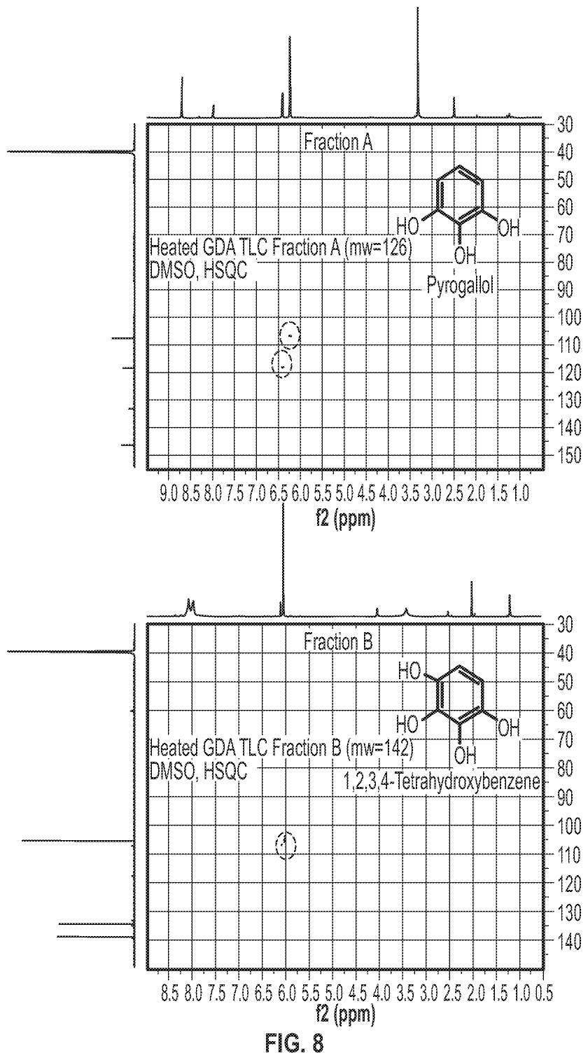

40 mL of parr reaction product with 14.5 w/v % GDA and 0.5 w/v % of glucose were incubated at 90° C. for 30 hrs to convert the GDA to 1,2,3,4-tetrahydroxybenzene. The incubated samples were extracted with 80 mL of ethyl acetate twice. The extracted samples were concentrated and run on TLC plates as shown in FIG. 7A. Two spots, A and B, were visualized on the plate. The fractions A and B were extracted from a preparative TLC plate and analyzed on LC/MS. The MS data, FIG. 7B, shows the molecular weight of fraction A and B are 126 and 142 respectively.

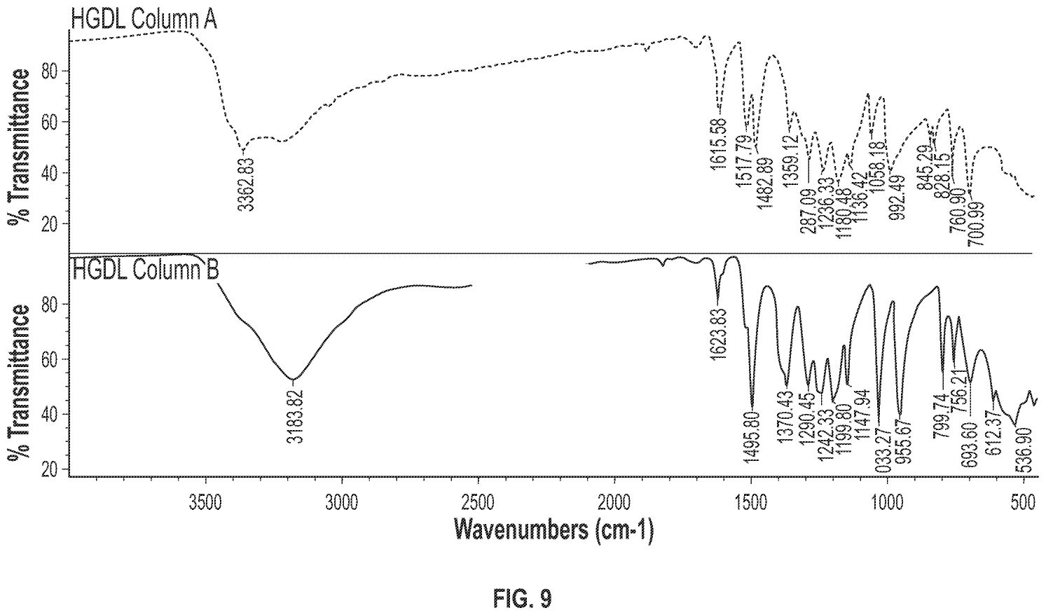

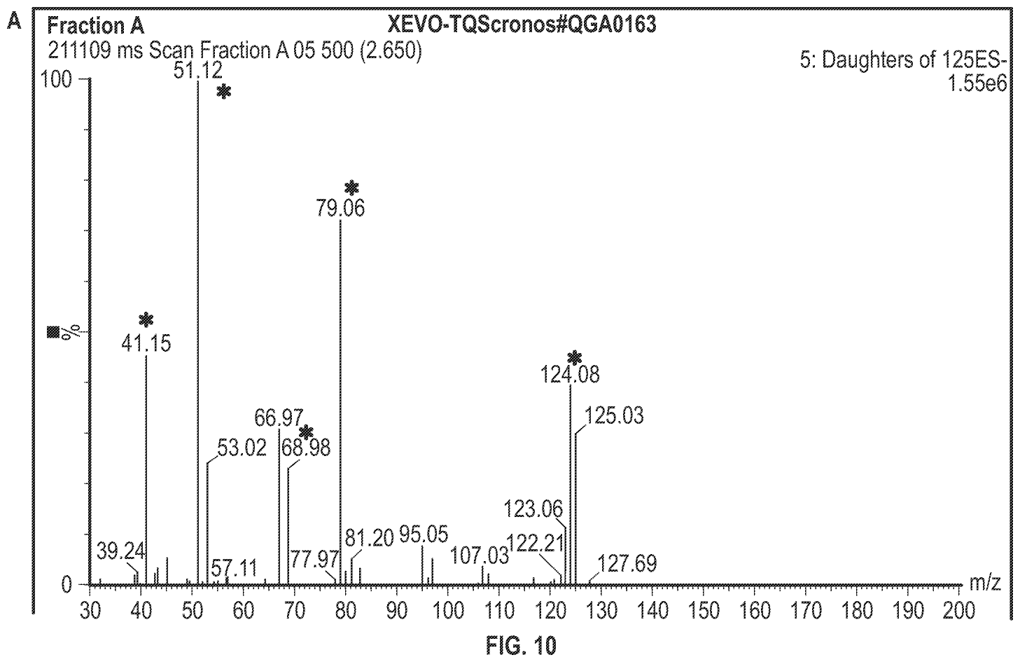

The purified fraction A and B were analyzed by 2D-NMR (HSQC, in DMSO-d6) to determine the molecular structures. The NMR data shown on FIG. 8 suggests the molecule in fraction A is 1,2,3-trihydroxybenzene, also called pyrogallol, and the molecule in fraction B is 1,2,3,4-tetrahydroxybenzene. FT-IR analysis (FIG. 9) shows no carbonyl function group on either of the molecules which further confirms the proposed molecular structure from the NMR analysis.

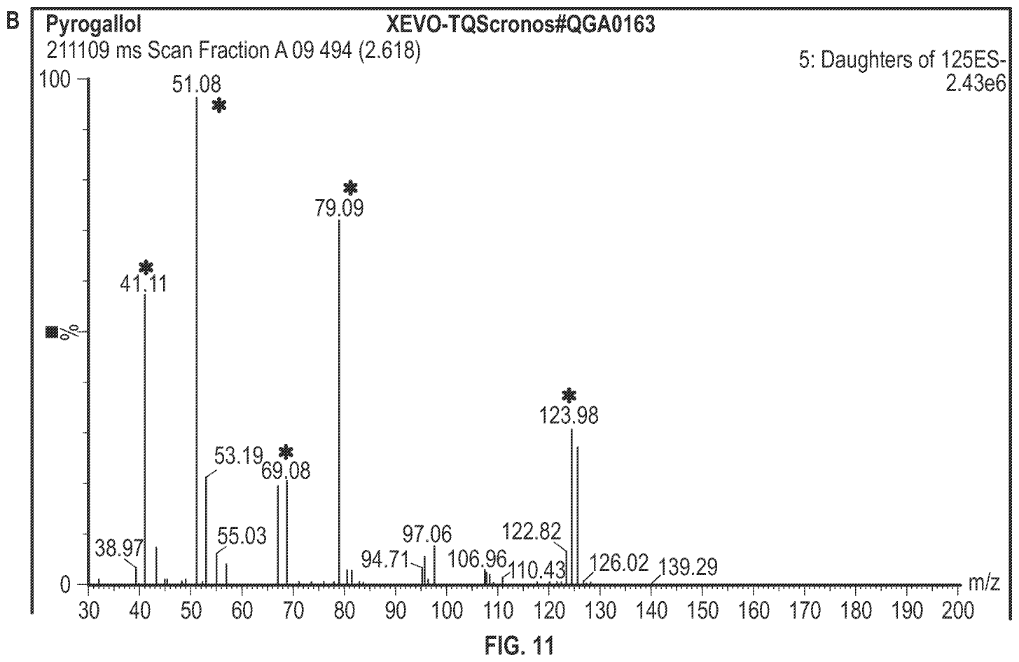

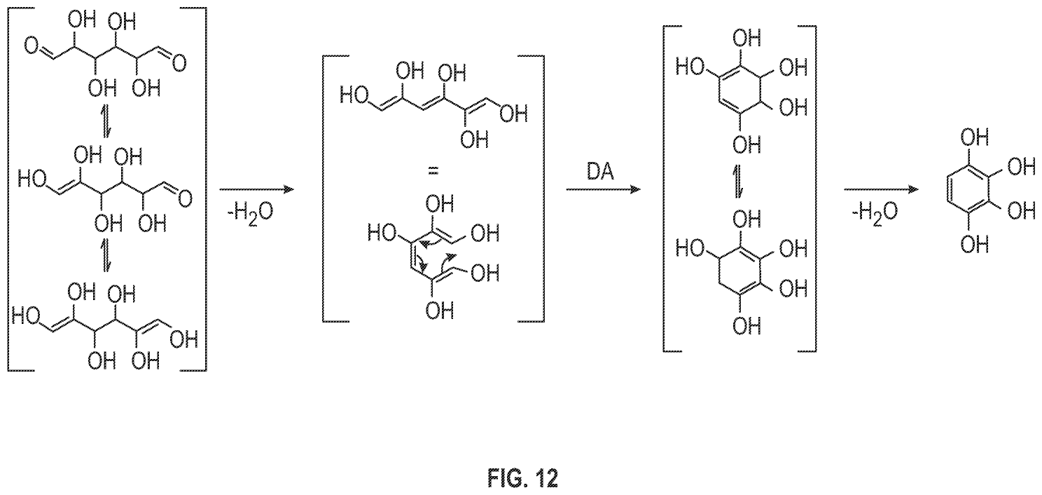

Further analysis was conducted with LC system that equipped with triple quadrupole mass detector and data is shown in FIGS. 10 and 11. The purified fraction A was analyzed with LC/QQQ and compared with the authentic standard pyrogallol. The fragmentation pattern of fraction A molecule perfectly matches the pyrogallol standard. A proposed reaction mechanism of pyrogallol and 1,2,3,4-tetrahydroxybenzene production from the GDA sample heat incubation is shown in FIG. 12.

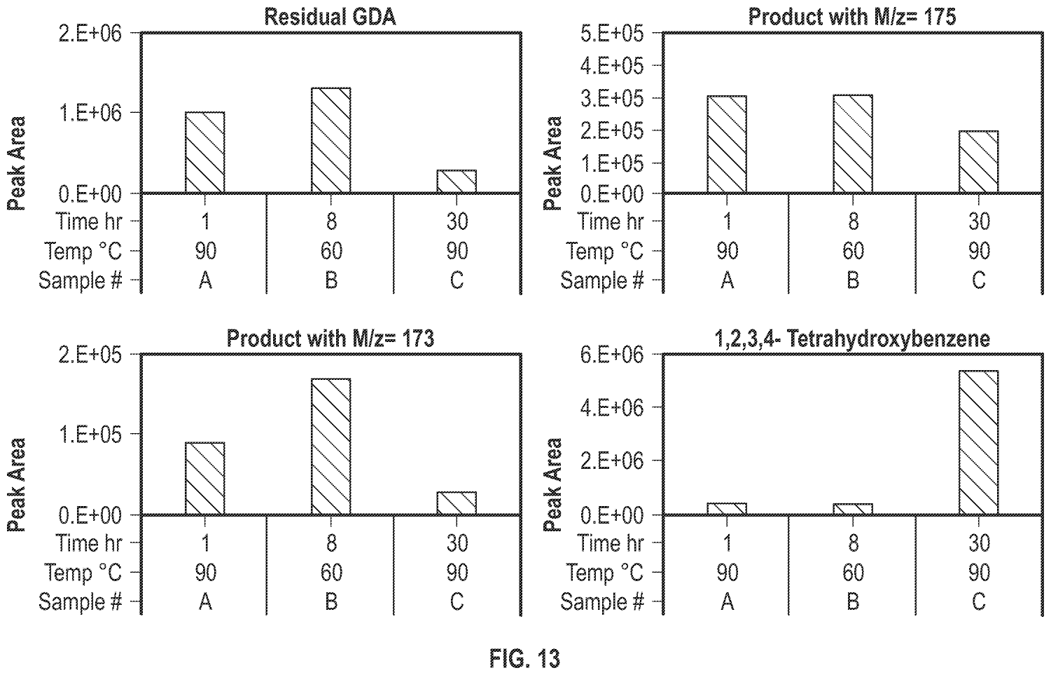

Three GDA samples generated from Parr reactions were incubated with different conditions, A) 90° C. for 1 hr, B) 60° C. for 8 hrs, or C) 90° C. for 30 hrs to maximize the conversion of GDA to either of two intermediate molecules or 1,2,3,4-tetrahydroxybenzene. As seen in FIG. 13, sample A has a relatively higher concentration of intermediate molecule with molecular weight of 176, and sample B has the highest concentration of the second of intermediate molecule with molecular weight of 174, and sample C has the highest conversion to 1,2,3,4-tetrahydroxybenzene.

All three different heat incubated GDA samples were tested as antimicrobial agents with several different industrially relevant bacteria. The General Aerobic Bacteria isolated and used for this testing were identified as Pseudomonas aeruginosa and Enterobacter aggolomerans. Wild strain Sulfate Reducing Bacteria and Acid Producing Bacteria isolated from the produced fluids was also used. They were placed in a microtiter plate, where a high throughput test can be performed to determine MIC (minimum inhibitor concentration). Samples were tested at the following doses (1000, 500, 250, 125, 62.5, 31.2, 15.6, 7.8, 3.9, and 1.95 ppm) and concentration was calculated based on the initial GDA concentration (15 w/v %). The results are summarized in Table 6.

The antimicrobial testing results shown in FIG. 13 summarizes the MIC of each sample for different bacteria. The MIC is determined after 24 hours of incubation for the bacterial plates. The concentration of the well that displays no growth after the appropriate incubation time is recorded as the MIC value for the specific formulation. Sample C which has highest concentration of 1,2,3,4-tetrahydroxybenzene has significantly lower MIC for all three bacteria tested, which suggests 1,2,3,4-tetrahydroxybenzene has good antimicrobial activity.

TABLE 6
Antimicrobial testing of three heat GDA samples.
Microtiter Study of antimicrobial activity

| | General | | SRB (Sulfate | | APB (Acid | |
|---|---|---|---|---|---|---|
| | Wild Strain | | Reducing | | Producing | |
| Biocide | Bacteria | | Bacteria) | | Bacteria) | |
| Heat GDA A | 10,000 | ppm | 10,000 | ppm | 10,000 | ppm |
| Heat GDA B | 10,000 | ppm | 10,000 | ppm | 10,000 | ppm |
| Heat GDA C | 2500 | ppm | 2500 | ppm | 1250 | ppm |
| Glutaraldehyde - 50% | 1000 | ppm | 500 | ppm | 500 | ppm |

The subject matter having been shown and described, modifications thereof can be made by one skilled in the art without departing from the spirit and teachings of the subject matter. The aspects described herein are exemplary only and are not intended to be limiting. Many variations and modifications of the subject matter disclosed herein are possible and are within the scope of the disclosed subject matter. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an aspect of the present disclosure. Thus, the claims are a further description and are an addition to the aspects of the present invention. The discussion of a reference herein is not an admission that it is prior art to the presently disclosed subject matter, especially any reference that may have a publication date after the priority date of this application. The disclosures of all patents, patent applications, and publications cited herein are hereby incorporated by reference, to the extent that they provide exemplary, procedural or other details supplementary to those set forth herein.

1. A chemoenzymatic method of producing a polyhydroxybenzene, the method comprising:
contacting glucose with one or more biocatalysts under conditions suitable for the formation of D-glucodialdose; and
thermally treating D-glucodialdose in the presence of a dehydration catalyst under conditions suitable for the formation of the polyhydroxybenzene.

2. The method of claim 1, wherein the polyhydroxybenzene comprises 1,2,3,4-tetrahydroxybenzene.

3. The method of claim 1, wherein the biocatalyst is a member of the copper radical oxidase family.

4. The method of claim 1, wherein the one or more biocatalysts comprise a galactose oxidase.

5. The method of claim 1, wherein the one or more biocatalysts comprise a mutated galactose oxidase.

6. The method of claim 1, wherein the one or more biocatalysts has any of SEQ ID No.1 through SEQ ID No.:9.

7. The method of claim 1, wherein the one or more biocatalysts comprises a catalase.

8. The method of claim 1, wherein thermally treating comprises subjecting D-glucodialdose to a temperature ranging from about 40° C. to about 100° C.

9. The method of claim 1, wherein the dehydration catalyst comprises copper, manganese, a polyanion or a combination thereof.

10. The method of claim 1, further comprising recovering 1,2,3,4-tetrahydroxybenzene.

11. The method of claim 1, further comprising purifying 1,2,3,4-tetrahydroxybenzene.

12. The method of claim 1, wherein the purity of 1,2,3,4-tetrahydroxybenzene ranges from about 70% to equal to or greater than about 90%.

13. The method of claim 1, wherein the yield of 1,2,3,4-tetrahydroxybenzene ranges from about 50% to about 90%.

14. A chemoenzymatic method of producing 1,2,3,4-tetrahydroxybenzene comprising:
contacting glucose with a mutated copper radical oxidase and catalase under conditions suitable for the formation of D-glucodialdose; and
subjecting D-glucodialdose and a dehydration catalyst to temperatures ranging from about 40° C. to about 100° C. to form 1,2,3,4-tetrahydroxybenzene.

15. The method of claim 14, wherein the mutated copper radical oxidase comprises a galactose oxidase having any of SEQ ID No.7 through SEQ ID No.:9.

16. The method of claim 14, wherein the purity of 1,2,3,4-tetrahydroxybenzene ranges from about 70% to equal to or greater than about 90%.

17. The method of claim 14, wherein the yield of 1,2,3,4-tetrahydroxybenzene ranges from about 50% to about 90%.

18. The method of claim 14, wherein the method is carried out in a single reaction vessel.

19. The method of claim 14, further comprising contacting the 1,2,3,4-tetrahydroxybenzene with one or more pharmaceutically acceptable carriers or pharmaceutically acceptable diluents.

20. The method of claim 14, further comprising introducing the 1,2,3,4-tetrahydroxybenzene to industrial water.

21. The method of claim 14, wherein the industrial water comprises feed water.


SEQUENCE LISTING (9 sequences)

SEQ ID NO: 1 - protein, 639 aa, Fusarium graminearum
ASAPIGSAISRNNWAVTCDSAQSGNECNKAIDGNKDTFWHTFYGANGDPKPPHTYTIDMKTTQNVNGLSMLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPAAAAIEPTSGRVLMWSSYRNDAFGGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSWSGGVFEKNGEVYSPSSKTWTSLPNAKVNPMLTADKQGLYRSDNHAWLFGWKKGSVFQAGPSTAMNWYYTSGSGDVKSAGKRQSNRGVAPDAMCGNAVMYDAVKGKILTFGGSPDYQDSDATTNAHIITLGEPGTSPNTVFASNGLYFARTFHTSVVLPDGSTFITGGQRRGIPFEDSTPVFTPEIYVPEQDTFYKQNPNSIVRVYHSISLLLPDGRVFNGGGGLCGDCTTNHFDAQIFTPNYLYNSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

SEQ ID NO: 2 - protein, 680 aa, Fusarium graminearum
MKHFLSLALCFSSINAVAVTVPHKSGGTGSPEGSLQFLSLRASAPIGSAISRNNWAVTCDSAQSGNECNKAIDGNKDTFWHTFYGANGDPKPPHTYTIDMKTTQNVNGLSMLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAFGGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSWSGGVFEKNGEVYSPSSKTWTSLPNAKVNPMLTADKQGLYRSDNHAWLFGWKKGSVFQAGPSTAMNWYYTSGSGDVKSAGKRQSNRGVAPDAMCGNAVMYDAVKGKILTFGGSPDYQDSDATTNAHIITLGEPGTSPNTVFASNGLYFARTFHTSVVLPDGSTFITGGQRRGIPFEDSTPVFTPEIYVPEQDTFYKQNPNSIVRVYHSISLLLPDGRVFNGGGGLCGDCTTNHFDAQIFTPNYLYNSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQ

SEQ ID NO: 3 - protein, 605 aa, Aspergillus niger
MQTLLVSSLVVSLAAALPHYIRSNGIEASLLTDPKDVSGRTVDYIIAGGGLTGLTTAARLTENPNISVLVIESGSYESDRGPIIEDLNAYGDIFGSSVDHAYETVELATNNQTALIRSGNGLGGSTLVNGGTWTRPHKAQVDSWETVFGNEGWNWDNVAAYSLQAERARAPNAKQIAAGHYFNASCHGTNGTVHAGPRDTGDDYSPIVKALMSAVEDRGVPTKKDFGCGDPHGVSMFPNTLHEDQVRSDAAREWLLPNYQRPNLQVLTGQYVGKVLLSQNGTTPRAVGVEFGTHKGNTHNVYAEHEVLLAAGSAVSPTILEYSGIGMKSILEPLGIDTVVDLPVGLNLQDQTTATVRSRITSAGAGQGQAAWFATFNETFGDYSEKAHELLNTKLEQWAEEAVARGGFHNTTALLIQYENYRDWIVNHNVAYSELFLDTAGVASFDVWDLLPFTRGYVHILDKDPYLHHFAYDPQYFLNELDLLGQAAATQLARNISNSGAMQTYFAGETIPGDNLAYDADLSAWTEYIPYHFRPNYHGVGTCSMMPKEMGGVVDNAARVYGVQGLRVIDGSIPPTQMSSHVMTVFYAMALKISDAILEDYASMQ

SEQ ID NO: 4 - protein, 354 aa, Humicola insolens
MAPKTSTFLASLTGAALVAAHGHVSHIIVNGVQYRNYDPTTDFYSGNPPTVIGWSALNQDNGFIEPNNFGTPDIICHKSAKPGGGHVTVRAGDKISIVWTPEWPESHVGPVIDYLAACNGDCETVDKTSLRFFKIDGAGYDAAAGRWAADALRANGNSWLVQIPADLKAGNYVLRHEIIALHGAANPNGAQAYPQCINIRVTGGGNNQPSGVPGTQLYKASDPGILFNPWVANPQYPVPGPALIPGAVSSIPQSRSTATATGTATRPGADTDPTGVPPVVTTTSAPAQVTTTTSSRTTSLPQITTTFATSTTPPPPAATQSKWGQCGGNGWTGPTVCAPGSSCNKLNDWYSQCI

SEQ ID NO: 5 - protein, 731 aa, Bos taurus
MPAEDIRHPEKQPNSSKGAMWVCLQLPVFLASVTLFEVAASDTIAQAASTTTISDAVSKVKIQVNKAFLDSRTRLKTTLSSEAPTTQQLSEYFKHAKGRTRTAIRNGQVWEESLKRLRRDTTLTNVTDPSLDLTALSWEVGCGAPVPLVKCDENSPYRTITGDCNNRRSPALGAANRALARWLPAEYEDGLALPFGWTQRKTRNGFRVPLAREVSNKIVGYLDEEGVLDQNRSLLFMQWGQIVDHDLDFAPETELGSNEHSKTQCEEYCIQGDNCFPIMFPKNDPKLKTQGKCMPFFRAGFVCPTPPYQSLAREQINAVTSFLDASLVYGSEPSLASRLRNLSSPLGLMAVNQEAWDHGLAYLPFNNKKPSPCEFINTTARVPCFLAGDFRASEQILLATAHTLLLREHNRLARELKKLNPHWNGEKLYQEARKILGAFIQIITFRDYLPIVLGSEMQKWIPPYQGYNNSVDPRISNVFTFAFRFGHMEVPSTVSRLDENYQPWGPEAELPLHTLFFNTWRIIKDGGIDPLVRGLLAKKSKLMNQDKMVTSELRNKLFQPTHKIHGFDLAAINLQRCRDHGMPGYNSWRGFCGLSQPKTLKGLQTVLKNKILAKKLMDLYKTPDNIDIWIGGNAEPMVERGRVGPLLACLLGRQFQQIRDGDRFWWENPGVFTEKQRDSLQKVSFSRLICDNTHITKVPLHAFQANNYPHDFVDCSTVDKLDLSPWASREN

SEQ ID NO: 6 - protein, 639 aa, synthetic construct
ASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPAAAAIEPTSGRVLMWSSYRNDAFEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYKSDNHAWLFGWKKGSVFQAGPSTAMNWYYTSGSGDVKSAGKRQSNRGVAPDAMSGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

SEQ ID NO: 7 - protein, 639 aa, synthetic construct
ASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPAAAAIEPTSGRVLMWSSYRNDAFEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYKSDNHAWLFGWKKGSVFQAGPSTAMNWYYTSGSGDVKSAGKRQSNRGVAPDAMSGNAVMYDAVKGKILTFGGSPDFEDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

SEQ ID NO: 8 - protein, 639 aa, synthetic construct
ASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPVAAAIEPTSGRVLMWSSYRNDRFEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYKSDNHAWLFGWKKGSVFQAGPSTAMNWYYTSGSGDVKSAGKRQSNRGVAPDAMSGNAVMYDAVKGKILTFGGSPHFEDSDATTNAHIITLGEPGTSPNTVFASNGLYFARTYHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

SEQ ID NO: 9 - protein, 638 aa, synthetic construct
ASAPIGSAIPRNNWAVTCDSAQSGNECNKAIDGNKDTFWHTFYGANGPKPPHTYTIDMKTTQNVSGLSVLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPVAAAIEPTSGRVLMWSSYRNDRFEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSAKTWTFLPNAKVNPMLTADKQGLYKSDNHAWLFGWKKGSVFQAGPSTAMNWYYTSGSGDVKSAGKRQSNRGVAPDAMSGNAVMYDAVKGKILTFGGSPHFEDSDATTNAHIITLGEPGTSPNTVFASNGLYFARTYHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ